(19) United States
(12) Patent Application Publication
(10) Pub. No.: US 2012/0090605 A1
(43) Pub. Date: Apr. 19, 2012

(54) MEDICATION DELIVERY DEVICE

(75) Inventors: Liam McDaid, County Donegal (IE); Barry Henderson, County Londonderry (GB); James Harkin, County Donegal (IE)

(73) Assignee: UNIVERSITY OF ULSTER, Coleraine, County Londerderry (UK)

(21) Appl. No.: 13/379,750

(22) PCT Filed: Jun. 22, 2010
(86) PCT No.: PCT/EP2010/003761

§ 371 Date: Dec. 21, 2011

(30) Foreign Application Priority Data
Jun. 22, 2009 (GB) 0910649.3

(51) Int. Cl. A61M 15/00; G01F 15/00; A61M 11/04

(52) U.S. Cl. 128/200.23; 73/198

(57) ABSTRACT

A medication delivery device for delivering a medicament by inhalation, said device comprising a housing (2) defining a chamber adapted to receive a metered dose of medication from a medication dispensing device, such as a pressurised metered dose inhaler canister or a dry powder inhaler, said chamber having an outlet (4) communicating with a mouthpiece of the device whereby said metered dose of medication can be withdrawn from the chamber via the mouthpiece by inhalation by a patient, said chamber being provided with a moveable wall (5) moveable between a first position and a second position, whereby said moveable wall is arranged to move from its first position to its second position as a patient withdraws a dose of medication from the chamber by inhalation to verify complete inhalation of said dose of medication.

FIG. 2

This invention relates to a medication delivery device for delivering a medicament by inhalation and in particular to a medicament delivery device for use with a pressurised metered dose inhaler canister, said device having means for verifying that a correct dose of medication has been taken.

Pressurised metered dose inhalers are commonly used to deliver drugs orally, in particular in relation to drugs used to treat asthma, such as cortico-steroids. Such devices comprise a pressurised canister containing a drug and a propellant, the canister having a metering valve for dispensing a metered dose of the drug from an orifice, said orifice communicating with a mouthpiece from which a metered dose of medication can be delivered when the canister is depressed to activate the metering valve.

Both children and many adults have difficulty using pressurised metered dose inhalers due to a lack of coordination of their breathing or other medical complications. The current solution to this problem is the use of a spacer connected between an outlet of the inhaler and a mouthpiece to define a closed chamber for receiving the medication dispensed from the inhaler, from which the medication can be subsequently inhaled via the mouthpiece provided at an outlet end of the spacer. The spacer acts as a holding chamber to retain the medication dispensed from the inhaler and helps to reduce the speed at which the aerosol enters the mouth. This makes it easier to use the inhaler and helps ensure that more of the medication gets into the lungs instead of just into the mouth or the air.

The aerosol medication may be inhaled from the spacer gradually, possibly in several inhalation stages, obviating the need to coordinate activation of the inhaler with the patient's breathing. However, this makes it difficult to ascertain whether or not the correct dose has been taken (i.e. whether or not all of the medicament has been inhaled from the chamber of the spacer). On a single dose of corticosteroid, for example, this may be of less relevance. However, over a longer period of time the patient may not be achieving an appropriate level of medication, possibly leading to asthmatic episodes or overdosing, which can cause serious complications, in particular for the young and elderly.

Recently there has been a move away from injecting drugs, with inhalation being a more favoured delivery method. Such drugs, for example insulin, may have more critical dosage requirements, leading to a desire for greater control and verification of the dosage delivered.

According to the present invention there is provided a medication delivery device for delivering a medicament by inhalation, said device comprising a housing defining a chamber adapted to receive a metered dose of medication from a medication dispensing device, such as a pressurised metered dose inhaler canister or a dry powder inhaler, said chamber having an outlet communicating with a mouthpiece of the device whereby said metered dose of medication can be withdrawn from the chamber via the mouthpiece by inhalation by a patient, said chamber being provided with a moveable wall moveable between a first position and a second position, whereby said moveable wall is arranged to move from its first position to its second position as a patient withdraws a dose of medication from the chamber by inhalation to verify complete inhalation of said dose of medication.

Preferably said moveable wall is adapted to be moved from its first position to its second position by means of the suction generated by a patient during inhalation of said dose of medication. Preferably said chamber defines an interior space bounded by said moveable wall, said interior space having a first volume when said moveable wall is in its first position and a second volume when said moveable is in its second position, said first volume being greater than said second volume.

In a preferred embodiment said device comprises an elongate tubular housing having a first end and a second end, said moveable wall being slidably mounted within the housing for sliding movement therealong such that the movable wall is located adjacent said first end of said housing when in its first position and is located adjacent said second end of the housing when in its second position, said chamber being defined by an interior space of said housing between one side of the moveable wall and said second end of the housing.

Preferably at least a portion of the housing is formed from a transparent material whereby the position of the moveable wall within the housing can be seen.

In one embodiment, said moveable wall may be provided with an aperture through which said metered dose of medication is delivered into the chamber. Preferably said aperture is located to be coincident with an inlet of the housing communicating with the outlet orifice of a medication dispensing device when the moveable wall is in its first position so that said metered dose of medication is delivered into the chamber through said aperture.

A vent opening may be provided in the first end of the housing to allow air to enter the housing as the moveable wall moves from its first position to its second position during inhalation of a dose of medication.

Preferably the moveable wall is adapted to be moved from its second position to its first position following inhalation of a dose of medication under the action of gravity, alternatively a manually operable return means may be provided for moving the moveable wall from its second to its first position.

In one embodiment said housing comprises a cylindrical tube, said moveable wall comprising a disc shaped member slidably mounted within the tube. Preferably said moveable wall is provided with a peripheral flange adapted to be in sliding contact with the inner surface of the housing to provide a seal against the inner wall of the housing and to maintain the moveable wall perpendicular to the longitudinal axis of the housing.

Alternatively the housing may be provided with an inlet in a side wall of the housing, preferably adjacent said second end of the housing, for delivering said metered dose of medication into the chamber.

A check valve may be provided between the chamber and the mouthpiece of the device allowing air and medication to pass out of the chamber via said mouthpiece while preventing flow from the mouthpiece into the chamber.

In one embodiment a sensor is provided for sensing the position of the moveable wall within the housing and/or for detecting when the moveable wall reaches said second position, indicating complete inhalation of a dose of medication. The sensor may be connected to a microprocessor adapted to provide an indication of the position of the moveable wall and/or the complete inhalation of a dose of medication. Said microprocessor may be provided with a memory unit whereby the amount of medication taken by the patient in a single dose and/or number of complete doses delivered by the device can be recorded. The microprocessor may also be provided with a clock whereby the time at which each dosage is taken can be recorded. A battery may be provided for powering the microprocessor and sensor. A display device, such as an LCD display, may be provided for displaying information related to the operation of the device, such as the number of doses delivered and/or to verify complete inhalation of a dose of medication. The microprocessor may be programmed to provide an accurate measurement of the dose taken based upon the movement of the moveable wall. In addition, the device may incorporate communication means whereby information, such as the amount of medication taken by the patient in a single dose and/or number of complete doses delivered by the device and/or the frequency and/or time at which each dose is taken, can be communicated to another electronic device (such as a mobile device or desktop PC), using either a wireless or wired medium.

Said sensor may comprise an optical sensor, a sonic sensor, a contact sensor or any other suitable sensing device for detecting the position of the moveable wall within the housing and/or for detecting when the moveable wall reaches the second end of the housing.

An embodiment of the present invention will now be described, by way of example only, with reference to the accompanying drawings, in which:—

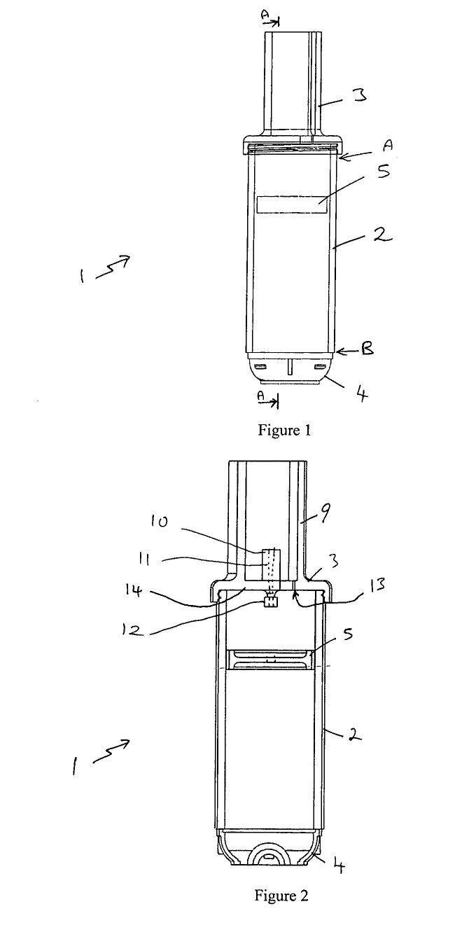

FIG. 1 is a perspective view of an aerosol medication delivery device according to an embodiment of the present invention;

FIG. 2 is a sectional view of the device of FIG. 1 along a line A-A of FIG. 1;

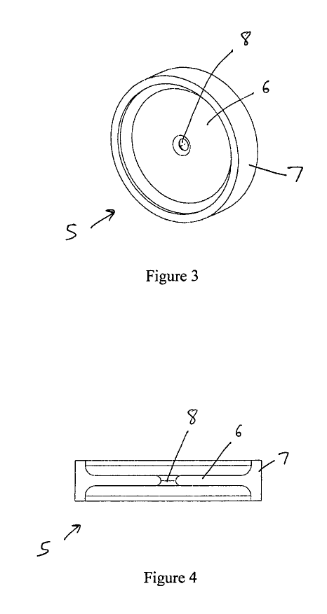

FIG. 3 is a perspective view of a moveable wall of the device of FIG. 1; and

FIG. 4 is a detailed perspective view of the moveable wall of FIG. 3.

As shown in the drawings, a medication delivery device 1 according to an embodiment of the present invention comprises a transparent cylindrical tubular housing 2 having a first moulded fitting 3 at a first end A, adapted to receive a pressurised metered dose inhaler canister for dispensing a metered dose of medication into the housing 2, and a further moulded fitting 4 at a second end B, providing an outlet for connection of a mouthpiece thereto.

A disc shaped moveable wall 5 is slidably mounted within the tubular housing 2 for sliding movement between the first and second ends A,B of the housing. As best seen from FIGS. 3 and 4, the moveable wall comprises a circular disc 6 having a peripheral flange 7 adapted to slidably engage the inner wall of the housing 2 to enable the moveable wall to slide within the housing between said first and second ends A, B thereof while providing a substantially air tight seal between the peripheral flange 7 of the wall 5 and the inner wall of the housing 2.

The peripheral flange 7 is of sufficient width to ensure that the wall 5 is maintained perpendicular to the axis of the housing 2 without tilting.

A central aperture 8 is provided within the disc, through which a dose of medicament may be delivered into a chamber defined within the housing from the pressurised metered dose inhaler canister, as will be described in more detail below.

As can be seen from FIG. 2, the first moulded fitting 3 is provided with a tubular holder portion 9 for receiving a pressurised metered dose inhaler canister, a female receiving portion 10 from receiving an outlet nozzle of the canister, said receiving portion communicating with a bore 11 terminating in an outlet nozzle 12 arranged coaxially within the housing 2 for delivering a metered dose of medication into the interior of the housing 2.

A vent opening 13 is provided in the first moulded fitting 3 to allow air to pass into and out of the housing 2 through an end wall 14 of the housing 2 defined by said first moulded fitting 3.

A check valve (not shown) may be fitted in the further moulded fitting 4 to allow air and medication to be drawn out of the housing 2 via the mouthpiece while prevent air from being blown or drawn into the housing 2 via the mouthpiece, as is known in the art.

In use, the device 1 is inverted from the orientation shown in FIGS. 1 and 2 and the moveable wall 5 is caused to move towards the first end A (i.e. towards end wall 14) of the housing 2 under the action of gravity until the moveable wall 5 rests adjacent the outlet nozzle 12.

The pressurised metered dose inhaler canister is pressed towards the housing 2, against the receiving portion 10, to activate the metering valve of the canister to dispense a metered dose of medication into the interior of the housing 2, through the aperture 8 in the moveable wall 2 and into a receiving chamber bounded by the inner wall of the housing 2, the moveable wall 5 and the further moulded fitting 4.

By dispensing a metered dose of medication through said aperture in the moveable wall 5 in a direction coincident with the longitudinal axis of the housing, the distance travelled by the aerosol medication before it impinges on a surface of the housing is maximised, minimising the risk of any deposition of medication on the inner walls of the housing.

The user then inhales through the mouthpiece to draw the aerosol medication from said chamber through the mouthpiece. As the user inhales, the suction caused by such inhalation causes the moveable wall 5 to advance along the housing 2 towards the second end B of the housing 2 (i.e. towards the mouthpiece) until the wall 5 reaches the second end B of the housing 2. The movement of the moveable wall 5 is clearly visible through the transparent housing 2 and thus provides a clear visual indication to verify when a complete dosage has been taken (i.e. when the moveable wall reaches the second end of the housing 2).

Once a complete dose has been taken, the moveable wall 5 can be returned to the first end A of the housing under the action of gravity by inverting the device 1 or by shaking the device 1.

The moveable wall 5 may comprise a brightly coloured plastic moulding to be readily visible though the side wall of the housing 2.

In an alternative embodiment, a sensor, such as a sonic or optical sensor, may be provided for determining the position of the moveable wall 5, such sensor being connected to a display device for displaying the position of the moveable wall. In one embodiment, the sensor may detect when the moveable wall 5 reaches the second end B of the housing 2 to indicate that a complete dose has been inhaled by the user. Alternatively the sensor may detect the position of the moveable wall 5 with respect to the housing 2 at any point within the housing 2 such that the dose delivered as the moveable wall is drawn along the housing can be determined. A microprocessor may be provided for establishing the dose taken based upon the change of position of the moveable wall 5.

Where a microprocessor is provided for connection to the sensor, the microprocessor may be provided with a storage device for recording the operation of the device 1, for example for recording the total number of complete doses taken and/or for recording the dosage taken in each dosing operation and/or the total dosage taken over time. Preferably the microprocessor includes a clock whereby the time at which each dosage is taken can be recorded.

In addition, the microprocessor may have the capability to communicate information to another electronic device (e.g. mobile device or desktop PC) using either a wireless or wired medium. Said electronic device may have the capability to host a software application whereby the recorded information or a variation of it can be displayed for home, mobile or professional use.

The sensor may comprise any sonic or optical device for determining the position of the moveable wall 5.

In an alternative embodiment the medication delivery device may be adapted for use with a dry powder inhaler. The inlet of the chamber may be modified to allow the dry powder to enter the chamber.

The invention is not limited to the embodiment(s) described herein but can be amended or modified without departing from the scope of the present invention.

1. A medication delivery device for delivering a medicament by inhalation, said device comprising,
a housing defining a chamber adapted to receive a metered dose of a medication from a medication dispensing device, said chamber having an outlet;
a mouthpiece in communication with said outlet of said chamber whereby the metered dose of medication can be withdrawn from said chamber via said mouthpiece by inhalation by a patient;
a moveable wall provided in said chamber, said moveable wall being moveable between a first position and a second position; and
wherein said moveable wall is arranged to move from its first position to its second position as a patient withdraws the dose of medication from said chamber by inhalation to verify complete inhalation of the dose of medication.

2. A device as claimed in claim 1, wherein said moveable wall is adapted to be moved from its first position to its second position by suction generated by the patient during inhalation of the dose of medication.

3. A device as claimed in claim 1, wherein said chamber defines an interior space bounded by said moveable wall, said interior space having a first volume when said moveable wall is in its first position and a second volume when said moveable wall is in its second position, said first volume being greater than said second volume.

4. A device as claimed in claim 1, wherein said housing comprises an elongate tubular housing having a first end and a second end, said moveable wall being slidably mounted within said housing for sliding movement therealong such that said movable wall is located adjacent said first end of said housing when in its first position and is located adjacent said second end of said housing when in its second position, said chamber being defined by an interior space of said housing between one side of said moveable wall and said second end of said housing.

5. A device as claimed in claim 1, wherein at least a portion of said housing comprises a transparent material whereby the position of said moveable wall within said housing can be seen through the transparent material of said housing.

6. A device as claimed in claim 1, wherein said moveable wall comprises an aperture through which the metered dose of medication is delivered into said chamber.

7. A device as claimed in claim 6, wherein said aperture is located to be coincident with an inlet of said housing, which is in communication with said outlet of said chamber when said moveable wall is in its first position so that the metered dose of medication is delivered into said chamber through said aperture.

8. A device as claimed in claim 1, further comprising a vent opening adapted to allow air to enter said housing as said moveable wall moves from its first position to its second position during inhalation of the dose of medication.

9. A device as claimed in claim 1, wherein said moveable wall is adapted to be moved from its second position to its first position due to gravity following inhalation of the dose of medication.

10. A device as claimed in claim 1, further comprising a manually operable return means for moving said moveable wall from its second position to its first position.

11. A device as claimed in claim 1, wherein said housing comprises a cylindrical tube and said moveable wall comprises a disc shaped member slidably mounted within said cylindrical tube.

12. A device as claimed in claim 11, wherein said moveable wall comprises a peripheral flange adapted to be in sliding contact with an inner surface of said housing to provide a seal against said inner surface of said housing and to maintain said moveable wall perpendicular to a longitudinal axis of said housing.

13. A device as claimed in claim 1, further comprising a check valve between said chamber and said mouthpiece of the device, said check valve configured to allow air and medication to pass out of said chamber via said mouthpiece while substantially preventing flow from said mouthpiece into said chamber.

14. A device as claimed in claim 1, further comprising a sensor for sensing the position of said moveable wall within said housing or for detecting when said moveable wall reaches said second position, indicating substantially complete inhalation of the dose of medication.

15. A device as claimed in claim 14, wherein said sensor is in communication with a microprocessor adapted to provide an indication of the position of said moveable wall or the complete inhalation of the dose of medication.

16. A device as claimed in claim 15, wherein said microprocessor comprises a memory unit for recording the amount of medication taken by the patient in a single dose or a number of complete doses delivered by the device.

17. A device as claimed in claim 15, further comprising a battery for powering said microprocessor and sensor.

18. A device as claimed in claim 14, further comprising a display device is provided for displaying information related to the operation of the device.

19. A device as claimed claim 14, wherein said sensor comprises one or more of an optical sensor, a sonic sensor, or a contact sensor for detecting the position of said moveable wall within said housing or for detecting when said moveable wall reaches said second position.

20. A device as claimed in claim 1, wherein said chamber defined by said housing is adapted to receive the metered dose of the medication from a medication dispensing device comprising one of a pressurised metered dose inhaler canister or a dry powder inhaler.

21. A medication device as claimed in claim 18, wherein said display device is configured to display information indicative of the number of doses delivered or indicative of complete inhalation of a dose of medication.
